(19) United States
(12) Patent Application Publication
(10) Pub. No.: US 2022/0125705 A1
(43) Pub. Date: Apr. 28, 2022

(54) TOPICAL COMPOSITIONS FOR THERMAL PROTECTION AND METHODS OF MAKING THE SAME

(71) Applicant: Leardi Enterprises, LLC, West Chester, PA (US)

(72) Inventor: Michael LEARDI, West Chester, PA (US)

(21) Appl. No.: 17/436,873

(22) PCT Filed: Mar. 10, 2020
(86) PCT No.: PCT/US2020/021794

§ 371 Date: Sep. 7, 2021

Related U.S. Application Data
(60) Provisional application No. 62/817,235, filed on Mar. 12, 2019.
(60) Provisional application No. 62/840,777, filed on Apr. 30, 2019.
(60) Provisional application No. 62/978,636, filed on Feb. 19, 2020.

(51) Int. Cl. A61K 8/898; A61K 8/891; A61K 8/25; A61K 8/895; A61Q 17/04

(52) U.S. Cl. CPC A61K 8/898; A61K 8/891; A61Q 17/04; A61K 8/895; A61K 8/25

(57) ABSTRACT

Topical compositions for protecting the skin of a mammal from the transfer of heat from a surface are described herein. The compositions include a base formulation and additives. In some instances, the additives include thermally-insulating nanoparticles and/or microparticles, and one or more of a dimethicone, a cyclomethicone, and an amodimethicone. In some instances, the additives include a silicone acrylate emulsion, the silicone acrylate emulsion comprising a blend of about 30% of acrylates/polytrimethylsiloxy-methacrylate copolymer anionic emulsion and laureth-1 phosphate and, optionally, thermally-insulating nanoparticles and/or microparticles. In some instances, the additives include a 60% non-ionic emulsion of a polydimethylsiloxane/vinyl copolymer having a viscosity of greater than 120×10⁶ mm²/s at 0.01 Hz, and having C12-13 Pareth-23 and C12-13 Pareth-3 and, optionally, thermally-insulating nanoparticles and/or microparticles.

### CROSS-REFERENCE TO RELATED APPLICATIONS

This application claims the benefit of U.S. Provisional Application No. 62/817,235 filed Mar. 12, 2019, U.S. Provisional Application No. 62/840,777 filed Apr. 30, 2019, and U.S. Provisional Application No. 62/978,636 filed Feb. 19, 2020, the entire contents of each of which are incorporated herein by reference.

### FIELD OF THE DISCLOSURE

The present disclosure relates to topical compositions designed to protect the skin of a mammal. More specifically, present disclosure relates to topical compositions designed to prevent the burning or irritation of skin due to contact with high-temperature surfaces.

### BACKGROUND OF THE DISCLOSURE

Sunscreen compositions have long been applied topically to skin and hair to protect them from the damaging effects of the sun's radiation, especially against ultraviolet (UV) radiation. The damaging effects of sunlight exposure on skin are well documented and include increased incidence of skin carcinogenesis, pigmentation, anomalies and precancerous lesions such as actinic keratosis, melanoma and non-melanoma skin cancers, as well as accelerated skin aging. In recent years, a growing number of studies show that damage is caused not only by the UVB irradiation (290-320 nm), but also by UVA irradiation (320-400 nm).

Sunscreen compositions are commonly used during outdoor activity. Many people have occupations which require them to be exposed to the sun for long periods of time. Others choose to use their free time in outdoor recreations e.g. sunbathing, playing golf, surfing, fishing, skiing and swimming. All of these activities promote perspiration or allow the body to come in contact with water. Numerous sunscreen compositions have been developed which absorb ultraviolet light to protect the human body against this radiation, whether the source be from the sun or from manmade devices. These compositions incorporate ultraviolet absorbing agents that absorb one or both of UVA and UVB irradiation and should be resistant to removal from the skin by perspiration or water in order to broaden and prolong their effectiveness.

### BRIEF DESCRIPTION OF THE DRAWINGS

FIG. 1 is a graph showing thermal response data for topical composition formulations in accordance with various aspects of the present disclosure; and

FIG. 2 is a graph showing thermal response data for other topical composition formulations in accordance with various aspects of the present disclosure.

### DETAILED DESCRIPTION

The following description of the embodiments is merely exemplary in nature and is in no way intended to limit the subject matter of the present disclosure, their application, or uses.

As used throughout, ranges are used as shorthand for describing each and every value that is within the range. Any value within the range can be selected as the terminus of the range. Unless otherwise specified, all percentages and amounts expressed herein and elsewhere in the specification should be understood to refer to percentages by weight.

For the purposes of this specification and appended claims, unless otherwise indicated, all numbers expressing quantities, percentages or proportions, and other numerical values used in the specification and claims, are to be understood as being modified in all instances by the term “about.” The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent, alternatively ±5 percent, and alternatively ±1 percent of the given numeric value provided such a deviation does not alter the end function or result of the value. Accordingly, unless indicated to the contrary, the numerical parameters set forth in this specification and attached claims are approximations that can vary depending upon the desired properties sought to be obtained by the present invention.

It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. For example, as used in this specification and the following claims, the terms “comprise” (as well as forms, derivatives, or variations thereof, such as “comprising” and “comprises”), “include” (as well as forms, derivatives, or variations thereof, such as “including” and “includes”) and “has” (as well as forms, derivatives, or variations thereof, such as “having” and “have”) are inclusive (i.e., open-ended) and do not exclude additional elements or steps. Accordingly, these terms are intended to not only cover the recited element(s) or step(s), but may also include other elements or steps not expressly recited. Furthermore, as used herein, the use of the terms “a” or “an” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

While sunscreen compositions have long been known to help protect the skin of a mammal from harmful UVA and UVB irradiation, there does not appear to be a similar composition designed with the specific purpose of protecting skin from the direct transfer of heat via conduction. That is, there does not appear to be topical compositions designed to prevent the direct transfer of heat from a surface to the skin of a mammal.

The present disclosure is directed to topical compositions that have the primary purpose of protecting the skin of a mammal from burns or other heat-induced irritation arising from heat transfer from surfaces to the skin. The topical compositions can also have secondary purposes such as being a UV absorber/blocker, a moisturizer, a rejuvenator, a cleanser, a vitamin C serum, a growth factor serum, a peptide serum, an acne treatment, a liquid skin adhesive (“liquid stitches”), a pain reliever (joints, muscles, burns, cuts, scraps, and so on), an antimicrobial, a bug repellant, or a cosmeceutical. Topical compositions according to the present disclosure can be in various forms. In some instances, topical compositions according to the present disclosure can be in the form of a cream or lotion. In some instances, topical compositions according to the present disclosure can be in the form of a gel. In some instances, topical compositions according to the present disclosure can be in the form of an aerosol.

In accordance with various aspects of the present disclosure, a topical composition for prevention of conductive heat transfer can include a base formulation, and one or more additives including a dimethicone or mixture of dimethicones, a cyclomethicone or mixture of cyclomethicones, and an amodimethicone or mixture of amodimethicones. Dimethicones are also commonly referred to as polydimethylsiloxanes having the following chemical formula:

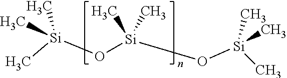

where n is an integer ranging from 0 to 500. In some instances, dimethicones can have molecular weights ranging from 162.38 to greater than about 500,000 g/mol. In general, dimethicones having molecular weights between about 162.38 and about 10,000 g/mol are preferable for use in topical compositions according to the present disclosure. More preferably, dimethicones having molecular weights between about 162.38 and about 5,000 g/mol are used in topical compositions according to the present disclosure. Even more preferably, dimethicones having molecular weights between about 162.38 and about 2,500 g/mol are used in topical compositions according to the present disclosure. Even more preferably, dimethicones having molecular weights between about 162.38 and about 1,000 g/mol are used in topical compositions according to the present disclosure. Cyclomethicones include hexamethylcyclotrisiloxane ([(CH₃)₂SiO]₃), octamethylcyclotetrasiloxane ([(CH₃)₂SiO]₄), decamethylcyclopentasiloxane ([(CH₃)₂SiO]₅), and dodecamethylcyclohexasiloxane ([(CH₃)₂SiO]₆). In some instances, a mixture of decamethylcyclopentasiloxane and dodecamethylcyclohexasiloxane can be used in topical compositions according to the present disclosure. Amodimethicones be described as exhibiting the following formula:

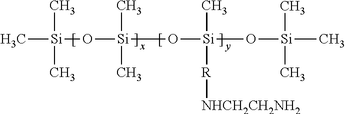

or the formula:

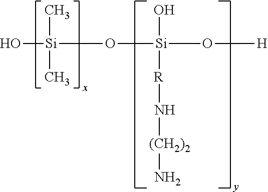

In both formulae, x any y are integers and R is —(CH₂)₃— or —CH₂CH(CH₃)CH—. In general, amodimethicones having molecular weights between about 5,000 and about 10,000 g/mol are preferable for use in topical compositions according to the present disclosure.

In some instances, the base formulation of the topical composition is a liquid. In other instances, the base formulation of the topical composition is a solid. In yet other instances the base formulation of the topical composition is a semisolid, a viscous formulation having the qualities of both a solid and a liquid. In yet other instances, the base formulation of the topical composition is an emulsion (droplets of a first liquid dispersed in a second liquid, where the first and second liquids are not miscible). In some instances, the base formulation of the topical composition can be a suspension (one or more solid particles dispersed throughout a bulk fluid). In some instances, the topical composition can include about 50 to about 95 volume percent (v/v %) of the base formulation. In other instances, the topical composition can include about 50 to about 80 v/v % of the base formulation. Preferably, the topical composition can include about 50 to about 75 v/v % of the base formulation and alternatively about 50 to about 70 v/v % of the base formulation. The topical composition can include between about 10 and about 60 v/v % of the one or more additives. In some instances, the topical composition includes between about 20 and about 60 v/v % of the one or more additives. In other instances, the topical composition includes between about 25 and about 55 v/v % of the one or more additives. In yet other instances, the topical composition includes between about 30 and about 50 v/v % of the one or more additives. In some instances, the one or more additives is only one of dimethicone, cyclomethicone and amodimethicone. In some instances, the one or more additives is a mixture of two of dimethicone, cyclomethicone and amodimethicone. In some instances, the one or more additives is a mixture of dimethicone, cyclomethicone and amodimethicone. In some instances, the one or more additives is a mixture of thermally insulating nano- and/or microparticles and only one of dimethicone, cyclomethicone and amodimethicone. In some instances, the one or more additives is a mixture of thermally insulating nano- and/or microparticles and two of dimethicone, cyclomethicone and amodimethicone. In some instances, the one or more additives is a mixture of thermally insulating nano- and/or microparticles, dimethicone, cyclomethicone and amodimethicone.

In accordance with various aspects of the present disclosure, a topical composition can include between about 1 and about 25 v/v % of a dimethicone or a mixture of dimethicones. In some instances, a topical composition can have between about 2 and about 15 v/v % of a dimethicone or a mixture of dimethicones. In other instances, a topical composition can have between about 3 and about 10 v/v % of a dimethicone or a mixture of dimethicones. In yet other instances, a topical composition can have between about 4 and about 10 v/v % of a dimethicone or a mixture of dimethicones. In yet other instances, a topical composition can have between about 5 and about 10 v/v % of a dimethicone or a mixture of dimethicones. In yet other instances, a topical composition can have between about 5 and about 8 v/v % of a dimethicone or a mixture of dimethicones. In yet other instances, a topical composition can have between about 5 and about 25 v/v % of a dimethicone or a mixture of dimethicones. In yet other instances, a topical composition can have between about 10 and about 25 v/v % of a dimethicone or a mixture of dimethicones.

In accordance with various aspects of the present disclosure, a topical composition can include between about 1 and about 25 v/v % of a cyclomethicone or a mixture of cyclomethicones. In some instances, a topical composition can have between about 2 and about 20 v/v % of a cyclomethicone or a mixture of cyclomethicones. In other instances, a topical composition can have between about 3 and about 15 v/v % of a cyclomethicone or a mixture of cyclomethicones. In yet other instances, a topical composition can have between about 5 and about 15 v/v % of a cyclomethicone or a mixture of cyclomethicones. In yet other instances, a topical composition can have between about 8 and about 12 v/v % of a cyclomethicone or a mixture of cyclomethicones. In yet other instances, a topical composition can have about 10 v/v % of a cyclomethicone or a mixture of cyclomethicones.

In accordance with various aspects of the present disclosure, a topical composition can include between about 1 and about 25 v/v % of an amodimethicone or a mixture of amodimethicones. In some instances, a topical composition can have between about 2 and about 15 v/v % of an amodimethicone or a mixture of amodimethicones. In other instances, a topical composition can have between about 3 and about 10 v/v % of an amodimethicone or a mixture of amodimethicones. In yet other instances, a topical composition can have between about 4 and about 8 v/v % of an amodimethicone or a mixture of amodimethicones. In yet other instances, a topical composition can have between about 4 and about 6 v/v % of an amodimethicone or a mixture of amodimethicones. In yet other instances, a topical composition can have about 5 v/v % of an amodimethicone or a mixture of amodimethicones.

In accordance with various aspects of the present disclosure, a topical composition can include between about 1 and about 20 w/w % of a silicone acrylate emulsion. In some instances, a topical composition can have between about 2 and about 17.5 w/w % of a silicone acrylate emulsion. In other instances, a topical composition can have between about 3 and about 15 w/w % of a silicone acrylate emulsion. In yet other instances, a topical composition can have between about 4 and about 12.5 w/w % of a silicone acrylate emulsion. In yet other instances, a topical composition can have between about 5 and about 10 w/w % of a silicone acrylate emulsion. In yet other instances, a topical composition can have about 5 w/w % of a silicone acrylate emulsion. In yet other instances, a topical composition can have about 10 w/w % of a silicone acrylate emulsion. In some instances, the silicone acrylate emulsion is a blend of about 30% of acrylates/polytrimethylsiloxy-methacrylate copolymer anionic emulsion having laureth-1 phosphate. In some instances, the silicone acrylate emulsion is a blend of about 40% of acrylates/polytrimethylsiloxy-methacrylate copolymer in 2 cSt polydimethylsiloxane.

In accordance with various aspects of the present disclosure, a topical composition can include between about 1 and about 20 w/w % of a divinyldimethicone/dimethicone copolymer emulsion or suspension. In some instances, a topical composition can have between about 2 and about 17.5 w/w % of a divinyldimethicone/dimethicone copolymer emulsion or suspension. In other instances, a topical composition can have between about 3 and about 15 w/w % of a divinyldimethicone/dimethicone copolymer emulsion or suspension. In yet other instances, a topical composition can have between about 4 and about 12.5 w/w % of a divinyldimethicone/dimethicone copolymer emulsion or suspension. In yet other instances, a topical composition can have between about 5 and about 10 w/w % of a divinyldimethicone/dimethicone copolymer emulsion or suspension. In yet other instances, a topical composition can have about 5 w/w % of a divinyldimethicone/dimethicone copolymer emulsion or suspension. In yet other instances, a topical composition can have about 10 w/w % of a divinyldimethicone/dimethicone copolymer emulsion or suspension. In some instances, a suitable divinyldimethicone/dimethicone copolymer emulsion is a 60% non-ionic emulsion of polydimethylsiloxane/vinyl copolymer having a viscosity of greater than 120×10⁶ mm²/s at 0.01 Hz. In some instances, a suitable divinyldimethicone/dimethicone cross-polymer suspension has a viscosity of about 6200 mPa·s or about 6700 mPa·s with a D₅₀ particle size of less than 4 micrometers. In some instances, the emulsions or suspensions further include C12-13 Pareth-23, C12-13 Pareth-3 and/or C12-14 Pareth 12.

In some instances, the one or more additives can be added to a base formulation such that the volumetric ratio of cyclomethicone(s) to the sum of the dimethicone(s) and amodimethicone(s) C: [D+A] in the topical composition equals about 1. In some instances, the one or more additives can be added to a base formulation such that the volumetric ratio (C: [D+A]) ranges from about 0.9:1 to about 1:1, alternatively 0.8:1 to about 1:1, alternatively from 0.7:1 to about 1:1, alternatively from 0.6:1 to about 1:1, alternatively from 0.5:1 to about 1:1, and alternatively from 0.25:1 to about 1:1. In some instances, the one or more additives can be added to a base formulation such that the volumetric ratio C: [D+A] ranges from about 1.1:1 to about 1:1, alternatively 1.2:1 to about 1.3:1, alternatively from 1.4:1 to about 1:1, alternatively from 1.5:1 to about 1:1, and alternatively from 2:1 to about 1:1.

In some instances, the one or more additives can be added to a base formulation such that the volumetric ratio of the dimethicone(s) and amodimethicone(s) (D:A) in the topical composition equals about 1. In some instances, the one or more additives can be added to a base formulation such that the volumetric ratio D:A ranges from about 0.9:1 to about 1:1, alternatively 0.8:1 to about 1:1, alternatively from 0.7:1 to about 1:1, alternatively from 0.6:1 to about 1:1, alternatively from 0.5:1 to about 1:1, and alternatively about 0.1:1 to 1:1. In some instances, the one or more additives can be added to a base formulation such that the volumetric ratio D:A ranges from about 1.1:1 to about 1:1, alternatively 1.2:1 to about 1.3:1, alternatively from 1.4:1 to about 1:1, alternatively from 1.5:1 to about 1:1, and alternatively from 2:1 to about 1:1.

In accordance with various aspects of the present disclosure, a particle-containing topical composition for prevention of conductive heat transfer can include a base formulation, and one or more of dimethicone, cyclomethicone, amodimethicone and thermally insulating nano- and/or microparticles.

In some instances, the base formulation of the particle-containing topical composition is a liquid. In other instances, the base formulation of the particle-containing topical composition is a solid. In yet other instances the base formulation of the particle-containing topical composition is a semisolid, a viscous formulation having the qualities of both a solid and a liquid. In yet other instances, the base formulation of the particle-containing topical composition is an emulsion (droplets of a first liquid dispersed in a second liquid, where the first and second liquids are not miscible). In some instances, the base formulation of the particle-containing topical composition can be a suspension (one or more solid particles dispersed throughout a bulk fluid). In some instances, the particle-containing topical composition can include about 40 to about 80 volume percent (v/v %) of the base formulation. In other instances, the particle-containing topical composition can include about 45 to about 70 v/v % of the base formulation. In some instances, the particle-containing topical compositions preferably include about 45 to about 65 v/v % of the base formulation and alternatively about 49 to about 65 v/v % of the base formulation. The particle-containing topical composition can include between about 5 and about 50 v/v % of the one or more additives. In some instances, the particle-containing topical composition includes between about 10 and about 55 v/v % of the one or more additives. In other instances, the particle-containing topical composition includes between about 15 and about 50 v/v % of the one or more additives. In yet other instances, the particle-containing topical composition includes between about 15 and about 45 v/v % of the one or more additives. In yet other instances, the particle-containing topical composition includes between about 20 and about 40 v/v % of the one or more additives. In some instances, the one or more additives is only one of dimethicone, cyclomethicone and amodimethicone, and thermally insulating nano- and/or microparticles. In some instances, the one or more additives is a mixture of two of dimethicone, cyclomethicone and amodimethicone, and thermally insulating nano- and/or microparticles. In some instances, the one or more additives is a mixture of dimethicone, cyclomethicone and amodimethicone, and thermally insulating nano- and/or microparticles. In some instances, the one or more additives is a mixture of a silicone acrylate emulsion and thermally insulating nano- and/or microparticles. In some instances, the one or more additives is a mixture of a divinyldimethicone/dimethicone copolymer emulsion or suspension and thermally insulating nano- and/or microparticles.

The thermally insulating nano- and/or microparticles can be any suitable nano- and/or microparticles known to one of skill in the art. In some instances, suitable nano- and/or microparticles can be made of metal oxides such as, for example, aluminum oxide (Al₂O₃), zinc oxide (ZnO), titanium oxide (TiO₂), silicon dioxide or silica (SiO₂), zirconium oxide (ZrO), zirconium dioxide (ZrO₂), magnesium oxide (MgO), calcium oxide (CaO), iron oxide (Fe₂O₃), an aluminosilicate, a borosilicate, sodium silicate, magnesium silicate, hydrated magnesium silicate, and magnesium aluminum silicate. In some instances, suitable nano- and/or microparticles can be made of insoluble ionic salts such as calcium carbonate (CaCO₃), calcium sulfate (CaSO₄), and barium sulfate (BaSO₄). In some instances, suitable nano- and/or microparticles can be made of polymers or resins such as, for example, a polystyrene (PS), a polymethylmethacrylate (PMMA), a polycarbonate (PC), a polyether ether ketone (PEEK), a polyacrylamide (PAM), a polyethylene terephthalate (PET), a low-density polyethylene (LDPE), a high-density polyethylene (HDPE), a polypropylene (PP), a polyisoprene (PI), aa polyvinyl chloride (PVC) a polychlorotrifluoroethylene (PCTFE), a poly-aramid, a polyacrylonitrile (PAN), a polyimide, and a polyester. In some instances, silica is preferred to be used as nano- or microparticles.

The thermally insulating nano- and/or microparticles can be any suitable shape. In some instances, thermally insulating nano- and/or microparticles that are spherical or substantially spherical in shape are preferred. The thermally insulating nano- and/or microparticles can have diameters generally ranging from about 1 nanometer to about 1000 micrometers, alternatively from about 5 nanometers to about 500 micrometers, alternatively from about 10 nanometers to about 100 micrometers, alternatively from about 15 nanometers to about 50 micrometers, alternatively from about 20 nanometers to about 10 micrometers, alternatively from about 25 nanometers to about 1 micrometer, alternatively from about 30 nanometers to about 500 nanometers, alternatively from about 30 nanometers to about 250 nanometers, alternatively from about 10 nanometers to about 250 nanometers, alternatively from about 10 nanometers to about 200 nanometers, alternatively from about 10 nanometers to about 150 nanometers, alternatively from about 10 nanometers to about 100 nanometers, alternatively from about 10 nanometers to about 70 nanometers, alternatively from about 1 micrometer to about 1000 micrometers, alternatively from about 10 micrometers to about 1000 micrometers, alternatively from about 50 micrometers to about 1000 micrometers, alternatively from about 100 micrometers to about 1000 micrometers, alternatively from about 250 micrometers to about 1000 micrometers, alternatively from about 500 micrometers to about 1000 micrometers, alternatively from about 1 micrometer to about 750 micrometers, alternatively from about 1 micrometer to about 500 micrometers, alternatively from about 1 micrometer to about 250 micrometers, and alternatively from about 1 micrometer to about 100 micrometers.

In accordance with various aspects of the present disclosure, a particle-containing topical composition can include between about 0.1 and about 25 v/v % of a dimethicone or a mixture of dimethicones. In some instances, a particle-containing topical composition can have between about 1 and about 20 v/v % of a dimethicone or a mixture of dimethicones. In other instances, a particle-containing topical composition can have between about 2 and about 15 v/v % of a dimethicone or a mixture of dimethicones. In yet other instances, a particle-containing topical composition can have between about 3 and about 15 v/v % of a dimethicone or a mixture of dimethicones. In yet other instances, a particle-containing topical composition can have between about 3 and about 10 v/v % of a dimethicone or a mixture of dimethicones. In yet other instances, a particle-containing topical composition can have between about 4 and about 10 v/v % of a dimethicone or a mixture of dimethicones. In yet other instances, a particle-containing topical composition can have between about 4 and about 8 v/v % of a dimethicone or a mixture of dimethicones. In yet other instances, a particle-containing topical composition can have between about 4 and about 7 v/v % of a dimethicone or a mixture of dimethicones.

In accordance with various aspects of the present disclosure, a particle-containing topical composition can include between about 1 and about 25 v/v % of a cyclomethicone or a mixture of cyclomethicones. In some instances, a particle-containing topical composition can have between about 2 and about 20 v/v % of a cyclomethicone or a mixture of cyclomethicones. In other instances, a particle-containing topical composition can have between about 3 and about 15 v/v % of a cyclomethicone or a mixture of cyclomethicones. In yet other instances, a particle-containing topical composition can have between about 5 and about 15 v/v % of a cyclomethicone or a mixture of cyclomethicones. In yet other instances, a particle-containing topical composition can have between about 5 and about 12.5 v/v % of a cyclomethicone or a mixture of cyclomethicones. In yet other instances, a particle-containing topical composition can have between about 5 and about 10 v/v % of a cyclomethicone or a mixture of cyclomethicones.

In accordance with various aspects of the present disclosure, a particle-containing topical composition can include between about 0.1 and about 20 v/v % of an amodimethicone or a mixture of amodimethicones. In some instances, a particle-containing topical composition can have between about 1 and about 15 v/v % of an amodimethicone or a mixture of amodimethicones. In other instances, a particle-containing topical composition can have between about 2 and about 10 v/v % of an amodimethicone or a mixture of amodimethicones. In yet other instances, a particle-containing topical composition can have between about 3 and about 8 v/v % of an amodimethicone or a mixture of amodimethicones. In yet other instances, a particle-containing topical composition can have between about 4 and about 8 v/v % of an amodimethicone or a mixture of amodimethicones.

In accordance with some aspects of the present disclosure, a particle-containing topical composition can include between about 0.1 and about 10 v/v % of thermally insulating nano- and/or microparticles. In some instances, a particle-containing topical composition can have between about 1 and about 10 v/v % of thermally insulating nano- and/or microparticles. In other instances, a particle-containing topical composition can have between about 2 and about 9 v/v % of thermally insulating nano- and/or microparticles. In yet other instances, a particle-containing topical composition can have between about 3 and about 8 v/v % of thermally insulating nano- and/or microparticles. In yet other instances, a particle-containing topical composition can have between about 4 and about 7 v/v % of thermally insulating nano- and/or microparticles.

In accordance with other aspects of the present disclosure, a particle-containing topical composition can include between about 5 and about 35 v/v % of thermally insulating nano- and/or microparticles. In some instances, a particle-containing topical composition can have between about 7.5 and about 30 v/v % of thermally insulating nano- and/or microparticles. In other instances, a particle-containing topical composition can have between about 10 and about 30 v/v % of thermally insulating nano- and/or microparticles. In yet other instances, a particle-containing topical composition can have between about 15 and about 30 v/v % of thermally insulating nano- and/or microparticles. In yet other instances, a particle-containing topical composition can have between about 20 and about 30 v/v % of thermally insulating nano- and/or microparticles.

In some instances, the one or more additives can be added to a base formulation such that the volumetric ratio of cyclomethicone(s) to the sum of the dimethicone(s) and amodimethicone(s) (C: [D+A]) in the particle-containing topical composition equals about 1. In some instances, the one or more additives can be added to a base formulation such that the volumetric ratio C:[D+A] ranges from about 0.9:1 to about 1:1, alternatively 0.8:1 to about 1:1, alternatively from 0.7:1 to about 1:1, alternatively from 0.6:1 to about 1:1, and alternatively from 0.5:1 to about 1:1. In some instances, the one or more additives can be added to a base formulation such that the volumetric ratio C: [D+A] ranges from about 1.1:1 to about 1:1, alternatively 1.2:1 to about 1.3:1, alternatively from 1.4:1 to about 1:1, and alternatively from 1.5:1 to about 1:1.

In some instances, the one or more additives can be added to a base formulation such that the volumetric ratio of the dimethicone(s) and amodimethicone(s) (D:A) in the particle-containing topical composition equals about 1. In some instances, the one or more additives can be added to a base formulation such that the volumetric ratio D:A ranges from about 0.9:1 to about 1:1, alternatively 0.8:1 to about 1:1, alternatively from 0.7:1 to about 1:1, alternatively from 0.6:1 to about 1:1, alternatively from 0.5:1 to about 1:1, and alternatively about 0.1:1 to 1:1. In some instances, the one or more additives can be added to a base formulation such that the volumetric ratio D:A ranges from about 1.1:1 to about 1:1, alternatively 1.2:1 to about 1.3:1, alternatively from 1.4:1 to about 1:1, alternatively from 1.5:1 to about 1:1, and alternatively from 2:1 to about 1:1.

In some instances, the one or more additives can be added to a base formulation such that the volumetric ratio of thermally insulating nano- and/or microparticles to the sum of the cyclomethicone(s), dimethicone(s) and amodimethicone(s) (Particles:[C+D+A]) in the particle-containing topical composition equals about 1. In some instances, the one or more additives can be added to a base formulation such that the volumetric ratio C:[D+A] ranges from about 0.9:1 to about 1:1, alternatively 0.8:1 to about 1:1, alternatively from 0.7:1 to about 1:1, alternatively from 0.6:1 to about 1:1, and alternatively from 0.5:1 to about 1:1. In some instances, the one or more additives can be added to a base formulation such that the volumetric ratio C: [D+A] ranges from about 1.1:1 to about 1:1, alternatively 1.2:1 to about 1.3:1, alternatively from 1.4:1 to about 1:1, and alternatively from 1.5:1 to about 1:1.

In some instances, a base formulation used in accordance with various aspects of the present disclosure can include one or more UVA and/or UVB sunscreen active agents. Suitable sunscreen active agents include. But are not limited to, avobenzone, homosalate, octisalate, octocrylene, oxybenzone, p-aminobenzoic acid (PABA), cinoxate, decamsule, dioxybenzone, menthyl anthranilate, octyl methoxycinnamate, octyl salicylate, octyl dimethyl PABA, phenylbenzimidazole sulfonic acid, sulisobenzone, triethanolamine salicylate, digalloyl trioleate, ethyl 4-[bis(hydroxypropyl)]aminobenzoate, glyceryl aminobenzoate, camphor benzalkonium methosulfate, terephthalidene dicamphor sulfonic acid, butyl methoxydibenzoylmethane, benzylidene camphor sulfonic acid, polyacrylamidomethyl benzylidene camphor, isoamyl p-methoxycinnamate, ethylhexyl triazone, drometrizole trielloxane, diethylhexyl butamido triazone, 4-methylbenzylidene camphor, 3-benzylidene camphor, ethylhexyl salicylate, ethylhexyl dimethyl PABA, benzophenone-4, methylene bis-benztriazolyl tetramethylbutylphenol, disodium phenyl dibenzimidazole tetrasulfonate, bis-ethylhexyloxyphenol methoxyphenol triazine, methylene bisbenzotriazolyl tetramethylbutylphenol, and bisethylhexyloxyphenol methoxyphenyl triazine, and mexenone.

In some instances, a base formulation used in accordance with various aspects of the present disclosure can include one or more cationic polymer to enhance the overall positive charge of one or more other components within the base formulation. Suitable cationic polymers include, but are not limited to, cationic cellulose ether derivatives, cationic guar gums, quaternary vinylpyrrolidone (PVP) copolymers, cationic polymers prepared with dimethyldiallylammonium chloride homopolymer or dimethyldiallylammonium chloride and acrylamide, quaterniums and polyquaterniums.

In some instances, a base formulation used in accordance with various aspects of the present disclosure can include one or more film forming agents to create a hydrophobic barrier layer on a user's skin. The one or more film forming agents can be used in conjunction with the one or more additives described above toward formation of a thermally protective topical composition. Suitable film forming agents include, but are not limited to, acrylic copolymers, butylated hydroxytoluene, lanolin derivatives, petrolatum, silicon derivatives, water-insoluble emollients, acacia gum, cellulose derivatives, guar derivatives, acrylate copolymers, acrylamide copolymers, acrylamide copolymers, acrylamide/sodium acrylate copolymers, acrylate/acrylamide copolymers, acrylate/ammonium methacrylate copolymers, acrylates/diacetoneacrylamide copolymers, acrylic/acrylate copolymers, adipic acid/dimethylaminohydroxypropyl diethlenetriamine copolymers, adipic acid/epoxypropyl/diethlenetriamine copolymers, albumen or salts or derivatives or serums thereof, allyl stearate/VA copolymers, aminoethylacrylate phosphate/acrylates copolymers, ammonium acrylates copolymers, ammonium alginate, ammonium vinyl acetate/acrylates copolymers, AMP acrylates/diacetoneacrylamide copolymers, balsams, starches, benzoic acid/phthalic anhydride/pentaerythritol/neopentyl glycol/palmitic acid copolymers, benzoin extract, butadiene/acrylonitrile copolymers, butylated urea-formaldehyde resins, butyl benzoic acid/phthalic anhydride trimethylolethane copolymers, butyl ester of ethylene maleic anhydride copolymers, butyl ester of PVM/MA copolymers, calcium/sodium PVM/MA copolymers, carboxymethyl hydroxyethyl cellulose, cellulose gum, collodion, copal, damar, diethylene glycolamine/epichlorohydrin/piperazine copolymers, dodecanedioic acid/cetearyl alcohol copolymers, ethylcellulose, ethylene/acrylate copolymers, ethylene/maleic anhydride copolymers, ethylene/vinyl acetate copolymers, dimethicone, hydroxybutyl methylcellulose, hydroxyethylcellulose, hydroxyethyl ethyl cellulose, hydroxypropylcellulose, hydroxypropyl guar, hydroxypropyl methylcellulose, isopropyl ester of PVM/MA copolymer, maltodextrins, melamine/formaldehyde resins, methacryloyl ethyl betaine methacrylates copolymers, nitrocellulose, octylacrylamide/acrylates/butylaminoethylmethacrylate copolymers, octylacrylamide/acrylates copolymers, phthalic anhydride/glycerin/gycidyl decanoate copolymers, polyacrylamides, polyacrylamidomethylpropane sulfone acids, polyacrylic acids, polybutylene terephthalates, polychlorotrifluoroethylenes, polyethylacrylates, polyethylenes, polyethylene terephthalates, polyisobutenes, polystyrenes, polyvinyl acetates, polyvinyl alcohols, polyvinyl butyrals, polyvinyl imidazolinium acetates, polyvinyl laurates, polyvinyl methyl ethers, calcium carrageenan, potassium carrageenan, magnesium carrageenan, sodium carrageenan, PVM/MA copolymer, polyvinylpyrrolidones (PVP), PVP/dimethylaminoethymethacrylate copolymers, PVP/eicosene copolymers, PVP/ethyl methacrylate/methacrylic acid copolymers, PVP/hexadecene copolymers, PVP/VA copolymers, PVP/vinyl acetate/itaconic acid copolymer, sodium acrylate/vinyl alcohol (VA) copolymers, sodium polymethacrylates, sodium polystyrene sulfonates, starch/acrylates/acrylamide copolymer, styrene/acrylate/acrylonitrile copolymers, styrene/acrylates/ammonium methacrylate copolymers, styrene/maleic anhydride copolymers, styrene/PVP copolymers, sucrose benzoate/sucrose acetate isobutyrate/butyl benzyl phthalate copolymers, vinyl acetate/crotonates copolymers, vinyl acetate/crotonic acid copolymers, and vinyl acetate/crotonic acid/vinyl neodecanoate copolymers.

In some instances, a base formulation used in accordance with various aspects of the present disclosure can include one or more surfactant metal complexes to enhance the reflective property of a sunscreen composition.

In some instances, a base formulation used in accordance with various aspects of the present disclosure can include one or more photostabilizing agents to prevent photodegradation of the topical composition.

In some instances, a base formulation used in accordance with various aspects of the present disclosure can include one or more analgesic or aesthetic agents. Suitable analgesic and aesthetic agents include, but are not limited to, dyclonine hydrochloride, aloe vera, diclofenac, capsaicin, benzalkonium chloride, butamben picrate, benzocaine, bupivacaine, calamine, chlorprocaine, cocaine, dibucaine, dyclonine, etidocaine, hexylcaine, ketamine, lidocaine, mepivacaine, menthol, procaine, pramoxine, prilocaine, phenol, tetracaine, xylocaine, and pharmaceutically acceptable salts thereof.

In some instances, a base formulation used in accordance with various aspects of the present disclosure can include one or more anti-acne agents. Suitable anti-acne agents include, but are not limited to, 5,7-dichloro-8-hydroxyquinoline, adapalene, azaleic acid, benzoyl peroxide, clindamycin, dapsone, erythromycin, long chain dicarboxylic acids, hydrocortisone, resorcinol, resorcinol acetate, salicylic acid, sulphur, tretinoin, urea, and zinc.

In some instances, a base formulation used in accordance with various aspects of the present disclosure can include one or more anti-allergenic agents. Suitable anti-allergenic agents include, but are not limited to, diphenhydramine, chlorpheniramine, tripelennamine, promethazine, clemastine, doxylamine, astemizole, terfenadine, loratadine, cimetadine, famotidine, nizatidine, ranitidine and cromolyn.

In some instances, a base formulation used in accordance with various aspects of the present disclosure can include one or more anti-cellulite agents. Suitable anti-cellulite agents include, but are not limited to, isobutylmethylxanthine, caffeine, theophylline, theobromine, aminophylline, yohimbine, minoxidil, diazoxide, N*-cyano-N-(tert-pentyl)-′-3-pyridinyl-guanidine, hormones, prostaglandins, diuretics, heat shock proteins, calcium channel blockers, immunosuppressant drugs, 5 alpha-reductase inhibitors, growth factors, tumor necrosis factors, retinoids, cytokines, cell adhesion molecules, glucocorticoids, botanical extracts, and antibiotics.

In some instances, a base formulation used in accordance with various aspects of the present disclosure can include one or more steroidal or non-steroidal anti-inflammatory agents. Suitable anti-inflammatory agents include, but are not limited to, corticosteroids, hydrocortisone, oxicams, salicylates, acetic acid derivatives, fenamates, propionic acid derivatives, pyrazoles, COX-2 inhibitors,

In some instances, a base formulation used in accordance with various aspects of the present disclosure can include one or more antioxidants. Suitable antioxidants include, but are not limited to, propyl, octyl and dodecyl esters of gallic acid, butylated hydroxyanisole (BHA), butylated hydroxytoluene (BHT), nordihydroguaiaretic acid, coenzyme Q-10, ascorbic acid and its salts, ascorbyl esters of fatty acids, ascorbic acid derivatives, tocopherol, tocopherol acetate, other esters of tocopherol, tocotrienols and their esters, and 6-hydroxy-2,5,7,8-tetramethylchroman-2-carboxylic acid, uric acid and its salts and alkyl esters, sorbic acid and its salts, lipoic acid, amines, sulfhydryl compounds, dihydroxy fumaric acid and its salts, lycine pidolate, arginine pidolate, nordihydroguaiaretic acid, bioflavonoids, curcumin, superoxide dismutase, silymarin, tea extracts, grape skin/seed extracts, melanin, and rosemary extracts.

In some instances, a base formulation used in accordance with various aspects of the present disclosure can include one or more anti-pruritic agents. Suitable anti-pruritic agents include, but are not limited to, alclometasone dipropionate, betamethasone valerate, and isopropyl myristate MSD.

In some instances, a base formulation used in accordance with various aspects of the present disclosure can include one or more anti-aging agents. In some instances, a base formulation used in accordance with various aspects of the present disclosure can include one or more anti-wrinkling agents. Suitable anti-aging or anti-wrinkling agents include, but are not limited to, hydroxy acids, retinal, retinoids, retinal palmitate, certain bicylic aromatic compounds, free-radical scavengers, keto acids, D- and L-amino acids, fatty acids, fatty alcohols, phospholipids, cholesterol, plant sterols, and fat-soluble vitamins.

In some instances, a base formulation used in accordance with various aspects of the present disclosure can include one or more anti-microbial agents. Suitable anti-microbial agents can include antifungal, antialgal, antibacterial, and antiseptic compounds.

In some instances, a base formulation used in accordance with various aspects of the present disclosure can include one or more anti-viral agents. Suitable anti-viral agents include, but are not limited to, acyclovir, penciclovir, metal salts (for example, silver nitrate, copper sulfate, and iron chloride) and organic acids (for example, malic acid, salicylic acid, succinic acid, and benzoic acid).

In some instances, a base formulation used in accordance with various aspects of the present disclosure can include one or more deodorants. Suitable deodorants include, but are not limited to, talcs, mono- or polyhalohydrates of aluminum and/or zirconium, aluminum or zirconium oxy salts or hydroxy salts or hydroxyhalides, bacteriostatic quaternary ammonium compounds, bioactive compounds, astringent salts, carbonate salts, and bicarbonate salts.

In some instances, a base formulation used in accordance with various aspects of the present disclosure can include one or more natural or synthetic dyes or colorants.

In some instances, a base formulation used in accordance with various aspects of the present disclosure can include one or more natural and/or synthetic fragrances.

In some instances, a base formulation used in accordance with various aspects of the present disclosure can include one or more essential oils.

In some instances, a base formulation used in accordance with various aspects of the present disclosure can include one or more moisturizing agents. Suitable moisturizing agents include, but are not limited to glycerin, chamomile, aloe, cetyl alcohol, grape seed oil, alpha hydroxy acids, silicone-based agents, petrolatum-based agents, and antioxidants

In some instances, a base formulation used in accordance with various aspects of the present disclosure can include one or more bug repellants agents.

In some instances, a base formulation used in accordance with various aspects of the present disclosure can include one or more thickening agents.

In some instances, a base formulation used in accordance with various aspects of the present disclosure can include one or more vitamins. Suitable vitamins include, but are not limited to, vitamin A, vitamin B, vitamin B2, vitamin C, vitamin D, vitamin E, and derivatives thereof such as tocopheryl acetate, beta-carotene, panthothenic acid vitamin E acetate and vitamin C palmitate.

In some instances, a base formulation used in accordance with various aspects of the present disclosure can include one or more lipids.

In some instances, a base formulation used in accordance with various aspects of the present disclosure can include one or more fatty acids. Suitable fatty acids include, but are not limited to, caprylic acid, capric acid, lauric acid, myristic acid, palmitic acid, stearic acid, arachidic acid, myristoleic acid, palmitoleic acid, sapienic acid, oleic acid, elaidic acid, vaccenic acid, linoleic acid, linoelaidic acid, a-linolenic acid, eicosapentaenoic acid, and docosahexaenoic acid.

In some instances, a base formulation used in accordance with various aspects of the present disclosure can include one or more amino acids. Suitable amino acids include, but are not limited to, glycine, L-taurine, N-acetyl L-cysteine (NAC), L-arginine, proline, sarcosine, alanine, β-alanine, and glutamine.

In some instances, a base formulation used in accordance with various aspects of the present disclosure can include one or more organic or metal complex dyes which functions as infrared absorbing agents.

In some instances, a base formulation used in accordance with various aspects of the present disclosure can include one or more cooling agents. Suitable cooling agents include, but are not limited to, menthol or isomers or derivatives thereof, menthone, peppermint oil, spearmint oil, WS-3, WS-23, menthyl succinate, menthyl lactate, and trimethylcyclohexanol.

In some instances, a base formulation used in accordance with various aspects of the present disclosure can include one or more heating agents. Suitable heating agents include, but are not limited to polyhydric alcohols, a capsicum powder, tincture or extract, capsaicin, homocapsaicin, homodihydrocapsaicin, nonanoyl vanillyl amide, nonanoic acid vanillyl ether, vanillyl alcohol alkyl ether derivatives, veratryl alcohol derivatives, substituted benzyl alcohol derivatives, substituted benzyl alcohol alkyl ethers, vanillin propylene glycol acetal, ethylvanillin propylene glycol acetal, ginger extract, ginger oil, gingeol, and gingeron.

In some instances, a base formulation used in accordance with various aspects of the present disclosure can include one or more preservatives. Suitable preservatives include, but are not limited to, citric acid, tartaric acid, phosphoric acid, iminodiacetic acid, nitrilotriacetic acid, hydroxyethyleneaminodiacetic acid and ethylenediaminetetraacetic acid and salts thereof, para-hydroxybenzoates (parabens); imidazolines, triclosan, hydantoins, and isothiazolidinones.

In some instances, a base formulation used in accordance with various aspects of the present disclosure can include one or more waxes. In some instances, a base formulation used in accordance with various aspects of the present disclosure can include one or more liquid hydrocarbons.

In some instances, a base formulation used in accordance with various aspects of the present disclosure can include one or more matrices. In some instance, the one or more matrices can include a fibrillary network. In some instances, the fibrillary network comprises polymeric fibers. In some instances, the fibrillary network comprises interfibrillary spaces. In some instances, the interfibrillary spaces can have a two-dimensional dimension ranging from about 1 to about 25 μm². In some instance, the one or more matrices can include a fibrillary mesh network. In some instances, the fibrillary network comprises polymeric fibers interwoven in a mesh configuration.

In some instances, a base formulation used in accordance with various aspects of the present disclosure can include one or more plasticizers. Plasticizers can include, but are not limited to, polyvinyl alcohol, hydroxypropyl methylcellulose, ethyl cellulose, and polyvinyl pyrrolidone.

In some instances, topical compositions according to the present disclosure are in the form of a stable emulsion. In some instances, topical compositions according to the present disclosure are in the form of a stable microemulsion. Emulsions or micoremulsions according to the present disclosure can include an emulsifier including, but not limited to a PVP/Eicosene copolymer, a sorbitan isostearate, an acrylates/C₁₀-C₃₀ alkyl acrylate crosspolymer, a PEG-20 sorbitan isostearate, and a combination of two or more thereof.

In some instances, a base formulation used in accordance with various aspects of the present disclosure can be made of avobenzone (about 3 w/w %), homosalate (about 13 w/w %), octisalate (about 5 w/w %), octocrylene (about 7 w/w %), oxybenzone (about 4 w/w %), sorbital (about 0.5-25.0 w/w %), aluminum starch octenyl succinate (about 0.1-12.0 w/w %), VP/eicosene copolymer (about 0.1-5.0 w/w %), stearic acid (about 0.5-15.0 w/w %), benzyl alcohol (about 0.1-5.0 w/w %), triethanolamine (about 0.1-5.0 w/w %), sorbitan isostearate (about 0.5-15.0 w/w %), tocopherol (about 0.01-5.0 w/w %), dimethicone (about 0.5-10.0 w/w %), polyglyceryl-3-distearate (about 0.5-10.0 w/w %), methyl paraben (about 0.01-1.0 w/w %), propyl paraben (about 0.01-1.0 w/w %), carbomer (about 0.1-5.0 w/w %), disodium EDTA (about 0.01-0.5 w/w %), and purified water (balance).

In some instances, a base formulation used in accordance with various aspects of the present disclosure can be made of avobenzone (about 3 w/w %), homosalate (about 15 w/w %), octisalate (about 5 w/w %), octocrylene (about 10 w/w %), oxybenzone (about 6 w/w %), butylene glycol (about 1-12.0 w/w %), microcrystalline cellulose (about 0.5-5.0 w/w %), butylate PVP (about 0.1-5.0 w/w %), behenyl alcohol (about 0.1-10.0 w/w %), benzyl alcohol (about 0.1-5.0 w/w %), glyceryl stearate (about 0.5-5.0 w/w %), palmitic acid (about 0.5-5.0 w/w %), myristyl alcohol (about 0.5-5.0 w/w %), tocopherol (about 0.01-5.0 w/w %), cellulose gum (about 0.1-3.0 w/w %), chlorphenesin (about 0.1-1.0 w/w %), cetyl alcohol (about 0.5-15.0 w/w %), lecithin (about 0.1-5.0 w/w %), disodium EDTA (about 0.01-0.5 w/w %), lauryl alcohol (about 0.5-5.0 w/w %), ascorbyl phosphate (about 0.1-5.0 w/w %), and purified water (balance).

In some instances, a base formulation used in accordance with various aspects of the present disclosure can be made of avobenzone (about 2 w/w %), homosalate (about 10 w/w %), octisalate (about 5 w/w %), octacrylene (about 5 w/w %), sorbitol (about 0.5-25.0 w/w %), ethylhexyl palmitate (about 0.5-10.0 w/w %), bis-stearyl ethylenediamine/neopentyl glycol/stearyl hydrogenated dimer dilinoleate copolymer (about 0.5-10.0 w/w %), benzyl alcohol (about 0.1-5.0 w/w %), dimethicone (about 0.5-10.0 w/w %), tocopherol (about 0.01-5.0 w/w %), cetyl phosphate (about 0.1-5.0 w/w %), aloe barbadensis leaf juice (about 0.5-10.0 w/w %), triethanolamine (about 0.1-5.0 w/w %), fragrance (about 0.1-5.0 w/w %), acrylates/C10-30 alkyl acrylate crosspolymer (about 0.5-10.0 w/w %), carbomer (about 0.1-5.0 w/w %), disodium EDTA (about 0.01-0.5 w/w %), chlorphenesin (about 0.1-1.0 w/w %) and purified water (balance).

### EXAMPLES

The following non-limiting examples are provided for illustrative purposes only in order to facilitate a more complete understanding of representative embodiments now contemplated.

These examples should not be construed to limit any of the embodiments described in the present specification.

### Example 1

For Example 1, a commercially available sunscreen (Coppertone® Tanning Defend & Glow™ SPF 15 sunscreen lotion, Bayer Healthcare LLC) and predetermined amounts of dimethicone (C₆H₁₈OSi₂, 162.38 g/mol, viscosity=500 cps, MakingCosmetics Inc., Redmond, Wash.), cyclomethicone (10-30 wt % decamethylcyclopentasiloxane [CAS # 541-02-6] and >60 wt % dodecamethylcyclohexasiloxane [CAS # 556-67-2], MakingCosmetics Inc., Redmond, Wash. and amodimethicone (amino-modified polydimethylsiloxane emulsion; 28-36 wt % amodimethicone [CAS # 68554-54-1], 1-3 wt % Trideceth-12 [CAS # 24938-91-5], 50-65 wt % water, and 1-3 wt % cetrimonium chloride [CAS # 112-02-7], Viscosity=5-50 cps [at 25° C.] MakingCosmetics Inc., Redmond, Wash.) were mixed to form various homogenous stable formulations. The general composition of the SPF-15 sunscreen is provided in Table 1, below. Some amounts in Table 1 are in the form of estimated ranges.

TABLE 1

| | Amount | | Amount |
|---|---|---|---|
| Ingredient | (wt %) | Ingredient | (wt %) |
| Avobenzone | 2 | tocopherol | 0.1-5 |
| | | (vitamin E) | |
| Homosalate | 10 | cetyl phosphate | 0.1-5 |
| Octisalate | 5 | aloe barbadensis | 0.5-10 |
| | | leaf extract | |
| Octocrylene | 5 | triethanolamine | 0.1-5 |
| Water | Remainder | fragrance | 0.1-5 |
| | to 100 | | |
| Sorbitol | 0.5-25 | acrylates/C10-30 | 0.5-10 |
| | | alkyl acrylate | |
| | | crosspolymer | |
| ethylhexyl palmitate | 0.5-10 | carbomer | 0.1-5 |
| bis-stearyl | 0.5-10 | disodium EDTA | 0.01-5 |
| ethylenediamine/ | | | |
| neopentyl | | | |
| glycol/stearyl | | | |
| hydrogenated | | | |
| dimerdilinoleate | | | |
| copolymer | | | |
| benzyl alcohol | 0.1-5 | chlorphenesin | 0.01-1 |
| dimethicone | 0.5-10 | | |

Each formulation was applied as layer at a thickness ranging from about 3-5 millimeters to a surface of a medical suturing practice skin (Suturing Doctor Suture Pads, 2 pc Set, FLESH SKIN-TONE COLOUR,) and allowed to set for approximately 20 seconds, forming a film thereon. The practice skin was then placed on a hot plate set to 60° C., the surface of the skin having the film thereon was placed on the hot plate. The temperature of the practice skin, on the surface opposite the hot plate, was monitored for 90 seconds, and starting and final temperatures were recorded. The results of Example 1 are displayed in Table 2, below.

TABLE 2

| | Sunscreen | | Temp Change |
|---|---|---|---|
| Ex. | (v/v %) | Additives (v/v %) | (%, 90 seconds) |
| 1 (21 trials) | 100 | 0 | 53.44 ± 2.80 |
| 2 (22 trials) | 80 | Dimethicone-5 | 45.82 ± 1.87 |
| | | Cyclomethicone-10 | |
| | | Amodimethicone-5 | |

As indicated in Table 2, use of the commercially available sunscreen resulted in a 53.44% increase in skin temperature over a 90 second period time. The addition of a mixture of dimethicone, cyclomethicone and amodimethicone (20 v/v % in total), resulted in a sunscreen formulation that exhibits only a 45.82% increase in skin temperature over the same period of time. The addition of dimethicone, cyclomethicone and amodimethicone therefore reduces the amount of heat transfer by about 14.3%.

### Example 2

For Example 2, a commercially available SPF-15 sunscreen (Table 1) and predetermined amounts of dimethicone (C₆H₁₈OSi₂, 162.38 g/mol, viscosity=500 cps, MakingCosmetics Inc., Redmond, Wash.), cyclomethicone (10-30 wt % decamethylcyclopentasiloxane [CAS # 541-02-6] and >60 wt % dodecamethylcyclohexasiloxane [CAS # 556-67-2], MakingCosmetics Inc., Redmond, Wash. and amodimethicone (amino-modified polydimethylsiloxane emulsion; 28-36 wt % amodimethicone [CAS # 68554-54-1], 1-3 wt % Trideceth-12 [CAS # 24938-91-5], 50-65 wt % water, ad 1-3 wt % cetrimonium chloride [CAS # 112-02-7], Viscosity=5-50 cps [at 25° C.] MakingCosmetics Inc., Redmond, Wash.) and silica gel beads (crushed to particles with diameters less than 1000 micrometers) were mixed to form various homogenous stable formulations.

Each formulation was applied as layer at a thickness ranging from about 3-5 millimeters to a surface of a medical suturing practice skin (Suturing Doctor Suture Pads, 2 pc Set, FLESH SKIN-TONE COLOUR,) and allowed to set for approximately 20 second, forming a film thereon. The practice skin was then placed on a hot plate set to 65° C., the surface of the skin having the film thereon was placed on the hot plate. The temperature of the practice skin, on the surface opposite the hot plate, was monitored for 90 seconds, and starting and final temperatures were recorded. The results of Example 2 are displayed in Table 3, below.

TABLE 3

| | Sunscreen | | Temp Change |
|---|---|---|---|
| Ex. | (v/v %) | Additives (v/v %) | (%, 90 seconds) |
| 1 (5 trials) | 100 | 0 | 58.08 ± 5.66 |
| 2 (5 trials) | 80 | Dimethicone-4 | 43.29 ± 2.01 |
| | | Cyclomethicone-8 | |
| | | Amodimethicone-4 | |
| | | Silica gel beads-4 | |
| 3 (5 trials) | 66.67 | Dimethicone-6.67 | 35.36 ± 2.00 |
| | | Cyclomethicone-13.33 | |
| | | Amodimethicone-6.67 | |
| | | Silica gel beads-6.67 | |

As indicated in Table 3, use of the commercially available sunscreen resulted in a 58.08% increase in skin temperature over a 90 second period time. The addition of a mixture of dimethicone, cyclomethicone, amodimethicone and silica gel (20 v/v % in total), resulted in a sunscreen formulation that exhibits only a 43.29% increase in skin temperature over the same period of time. The addition of dimethicone, cyclomethicone amodimethicone and silica (20 v/v % in total) therefore reduces the amount of heat transfer by about 25.5%. Furthermore, the addition of a mixture of dimethicone, cyclomethicone, amodimethicone and silica (33.33 v/v % in total), resulted in a sunscreen formulation that exhibits only a 35.36% increase in skin temperature over the same period of time. The addition of dimethicone, cyclomethicone, amodimethicone and silica (33.33 v/v % in total) therefore reduces the amount of heat transfer by about 39.1%.

### Example 3

In this example, various compositions according to the present disclosure are evaluated using a loss on drying technique, which is a modified version of <731> Loss on Drying by the United States Pharmacopeial (USP) convention, May 1, 2012. The equipment used in such evaluations are a balance, a desiccator, a 25±2° C./60±5% RH chamber and a 40±2° C./75±5% RH chamber. The modified version of the loss on drying technique was as follows.

First, an empty sample vial and a vial cap (unattached) were placed in a stability chamber under 25±5° C./60±5% RH or 40±5° C./75±5% RH for about 30 minutes. Second, the sample vial and vial cap were transferred to a desiccator to allow them to equilibrate to room temperature without attracting water from surrounding air. After equilibrium was obtained, about 2 grams of a sample composition was placed in the sample vial and the sample vial was capped with the vial cap. A rotational motion was applied to the sample vial to ensure the sample composition therein was of a substantially even height within the sample vial. The cap was then removed from the sample composition-containing sample vial and both were placed in a stability chamber (25±5° C./60±5% RH or 40±5° C./75±5% RH) for about 1 hour. After the 1 hour time period, the vial cap was placed on the sample composition-containing sample vial and transferred to a desiccator to allow for room temperature equilibration without attracting water from surrounding air. The weight of the sample composition was then measured to determine the amount of weight lost by the sample composition.

For Example 3, commercially available sunscreens (Coppertone® Ultra Guard SPF 50 sunscreen lotion and Coppertone® Water Babies SPF 50 sunscreen lotion, both by Bayer Healthcare LLC) and predetermined amounts of DOWSIL™ FA 4103 (a silicone acrylate emulsion being a blend of about 30% of acrylates/polytrimethylsiloxy-methacrylate copolymer anionic emulsion having laureth-1 phosphate) or DOWSIL™ HMW 2220 (a 60% non-ionic emulsion of polydimethylsiloxane/vinyl copolymer having a viscosity of greater than 120×10⁶ mm²/s at 0.01 Hz, having C12-13 Pareth-23 and C12-13 Pareth-3) were mixed to form various homogenous stable formulations. The general compositions of the SPF 50 sunscreens are provided in Table 4, below. Some amounts in Table 4 are in the form of estimated ranges.

TABLE 4

| | | | |
|---|---|---|---|
| | Amount | | Amount |
| Ingredient | (wt) | Ingredient | (wt) |

Sunscreen #1

| | | | |
|---|---|---|---|
| Avobenzone | 3 | Sorbitan | 0.5-15.0 |
| | | isostearate | |
| Homosalate | 13 | Dimethicone | 0.5-10.0 |
| Octisalate | 5 | Tocopherol | 0.01-5.0 |
| | | (Vitamin E) | |
| Octocrylene | 5 | Polyglycery1-3- | 0.5-10.0 |
| | | distrearate | |
| Oxybenzone | 4 | Methyl paraben | 0.01-1.0 |
| Water | Remainder | Propyl paraben | 0.01-1.0 |
| | to 100 | | |
| Sorbital | 0.5-25.0 | Carbomer | 0.1-5.0 |
| Aluminum starch | 0.1-12.0 | Disodium EDTA | 0.01-0.5 |
| octenyl succinate | | | |
| VP/eicosene | 0.1-5.0 | Triethanol amine | 0.1-5.0 |
| copolymer | | | |
| benzyl alcohol | 0.1-5.0 | | |

Sunscreen #2

| | | | |
|---|---|---|---|
| Avobenzone | 3 | Myristyl alcohol | 0.5-5.0 |
| Homosalate | 13 | Tocopherol | 0.01-5.0 |
| | | (Vitamin E) | |
| Octisalate | 5 | Cellulose Gum | 0.1-3.0 |
| Octocrylene | 10 | Chlorphenesin | 0.01-1.0 |
| Oxybenzone | 6 | Cetyl Alcohol | 0.5-15.0 |
| Water | Remainder | Lecithin | 0.1-5.0 |
| | to 100 | | |
| Butylene glycol | 1.0-12.0 | Disodium EDTA | 0.01-0.5 |
| microcrystalline | 0.5-5.0 | Lauryl alcohol | 0.5-5.0 |
| cellulose | | | |
| Butylate PVP | 0.1-5.0 | Ascorbyl | 0.1-1.0 |
| | | phosphate | |
| Behenyl alcohol | 0.1-10.0 | Glyceryl stearate | 0.5-5.0 |
| Benzyl alcohol | 0.1-5.0 | Palmitic acid | 0.5-5.0 |

The water loss (wt %) of various compositions according to Example 3 are provided in Table 5, below:

TABLE 5

| | | | Loss on Drying (%) | |
|---|---|---|---|---|
| | Sunscreen | Additive | 40 ± 5° C./ | 25 ± 5° C./ |
| Ex. | (#; w/w %) | (type; w/w %) | 75 ± 5% | 60 ± 5% |
| — | — | — | RH | RH |
| 1 | #1; 100 | 0 | 4.025 | 1.778 |
| 2 | #1; 95 | DOWSIL ™ FA | 3.135 | 0.731 |
| | | 4103; 5 | | |
| 3 | #1; 90 | DOWSIL ™ FA | 3.034 | 0.693 |
| | 4103; 10 | | | |
| 4 | #1; 95 | DOWSIL ™ | 3.116 | 0.674 |
| | | HMW 2220; 5 | | |
| 5 | #1; 90 | DOWSIL ™ | 3.051 | 0.681 |
| | | HMW 2220; 10 | | |
| 6 | #2; 100 | 0 | 3.754 | 0.652 |
| 7 | #2; 90 | DOWSIL ™ FA | 3.337 | 0.641 |
| | | 4103; 10 | | |
| 8 | #2; 90 | DOWSIL ™ | 3.942 | 0.621 |
| | | HMW 2220; 10 | | |

### Example 4

For each of the following formulations, a commercially available sunscreen (Coppertone® Tanning Defend & Glow™ SPF 15 sunscreen lotion, Bayer Healthcare LLC) and predetermined amounts of dimethicone (C₆H₁₈OSi₂, 162.38 g/mol, viscosity=500 cps, MakingCosmetics Inc., Redmond, Wash.), cyclomethicone (10-30 wt % decamethylcyclopentasiloxane [CAS # 541-02-6] and >60 wt % dodecamethylcyclohexasiloxane [CAS # 556-67-2], MakingCosmetics Inc., Redmond, Wash. and amodimethicone (amino-modified polydimethylsiloxane emulsion; 28-36 wt % amodimethicone [CAS # 68554-54-1], 1-3 wt % Trideceth-12 [CAS # 24938-91-5], 50-65 wt % water, and 1-3 wt % cetrimonium chloride [CAS # 112-02-7], Viscosity=5-50 cps [at 25° C.] MakingCosmetics Inc., Redmond, Wash.) and silica particles (crushed to particles with diameters less than 1000 micrometers) or boron nitride particles were mixed to form various homogenous stable formulations. The general composition of the SPF-15 sunscreen is provided in Table 1. Some amounts in Table 1 are in the form of estimated ranges. Table 6, below, summarizes the composition of each formulation prepared in this example.

TABLE 6

| Sample | SS (v/v %) | CY (v/v %) | DI (v/v %) | AM (v/v %) | SI (v/v %) | BN (v/v %) |
|---|---|---|---|---|---|---|
| Skin 0 (control) | — | — | — | — | — | — |
| A | 100.00 | — | — | — | — | — |
| B | 71.14 | 7.22 | 7.22 | 7.22 | 7.22 | — |
| C | 68.66 | 6.96 | 6.96 | 6.96 | 10.45 | — |
| D | 66.35 | 6.73 | 6.73 | 6.73 | 13.46 | — |
| Skin 1 (control) | — | — | — | — | — | — |
| E | 55.42 | 5.62 | 5.62 | 5.62 | 27.71 | — |
| F | 64.32 | 6.52 | 6.52 | 6.52 | 16.11 | — |
| Skin 2 (control) | — | — | — | — | — | — |
| G | 64.32 | 6.52 | 6.52 | 6.52 | — | 16.11 |
| H | 49.82 | 15.16 | 5.05 | 5.05 | 24.91 | — |
| I | 60.37 | 6.12 | 6.12 | 6.12 | 21.27 | — |

In Table 6, “SS” stands for sunscreen, “CY” stands for cylcomethicone, “DI” stands for dimethicone, “AM” stands from amodimethicone, “SI” stands for silica particles, and “BN” stands for boron nitrate particles. Additionally, each of “Skin 0,” “Skin 1,” and “Skin 2” stands for Vitro-Skin® (Florida Suncare Testing, Inc., IMS Division, Bunnell, Fla. 32110) without any formulation applied thereon.

In the following examples, skin sections are cut in 5 cm×5 cm squares and weighed. The coatings are applied to the skin in a realistic manner: Approximately 0.3 g of a formulation was rubbed into the skin section while ensuring an even distribution of the composition thereon. The weight of the skin section with sample was measured after application. The samples were then left to dry overnight (this was determined to be necessary as the skin analog is unable to absorb oily components of the compositions as a true skin would). After drying the samples were weighed once again to ensure similar amount of volatile loss and that the samples are ready for testing.

Film thickness is determined with a laser profilometer. Due to the transparency of the fake skin samples, a sheet of paper with a known thickness was placed on top of the samples for accurate interaction with the laser. The thickness of the skin and paper is then subtracted from the total height such that the thickness of the film layer was found. Table 7 presents the thickness of the skin and sample layers, where the sample thickness is determined by subtracting the measured skin thickness from the observed total height.

TABLE 7

| | | | Thermal | Thermal |
|---|---|---|---|---|
| | Film Thickness | Thickness² | Diffusivity | Diffusivity |
| Sample | (μm) | (mm²) | time (s) | (mm²/s) |
| Skin 0 | 0 | 0 | 2.05 | 0 |
| A | 33 | 1.1 | 2.34 | 0.47 |
| B | 287 | 82.4 | 5.32 | 15.47 |
| C | 235 | 55.2 | 3.07 | 18.00 |
| D | 220 | 84.4 | 3.09 | 15.68 |
| Skin 1 | 0 | 0 | 3.25 | 0 |
| E | 440 | 193.6 | 14.93 | 12.97 |
| F | 588 | 345.7 | 6.71 | 51.52 |
| Skin 2 | 0 | 0 | 3.48 | 0 |
| G | 172 | 29.6 | 4.29 | 6.89 |
| H | 352 | 123.9 | 12.20 | 10.16 |
| I | 541 | 292.9 | 11.63 | 25.19 |

Thermal testing was performed on an Accuplate hotplate, with a steel plate placed on top for thermal stability, set to 44° C. to simulate a hot surface. Temperature and time was recorded using a RDXL4SD Datalogger. A magnetic thermocouple was used to create a good thermal contact between the sample and the hotplate surface. Sample is placed film (i.e., composition) side down and the temperature was recorded for 5 min, which was seen to be sufficient time to reach thermal equilibrium. The amount of time taken for each skin and formulation-coated skin sample to reach 36° C. and 44° C. from room temperature is provided in Table 8, below.

TABLE 8

| Sample | 36° C. (seconds) | 44° C. (seconds) |
|---|---|---|
| Skin | 0.6 | 2.0 |
| A | 0.8 | 2.6 |
| B | 2.4 | 11.4 |
| C | 1.1 | 4.5 |
| D | 1.1 | 3.6 |
| Skin 1 | 1 | 15.0 |
| E | 10 | 324.0 |
| F | 4 | 196.0 |
| Skin 2 | 1 | 4.3 |
| G | 0.8 | 1.6 |
| H | 4 | 157.8 |
| I | 3.8 | 90 |

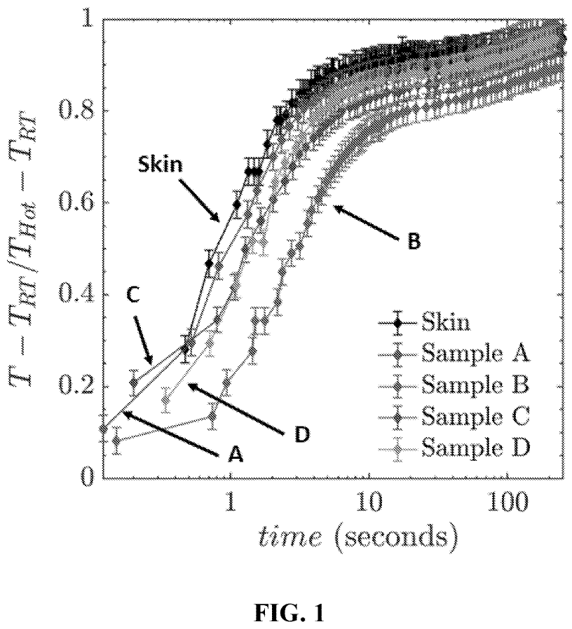

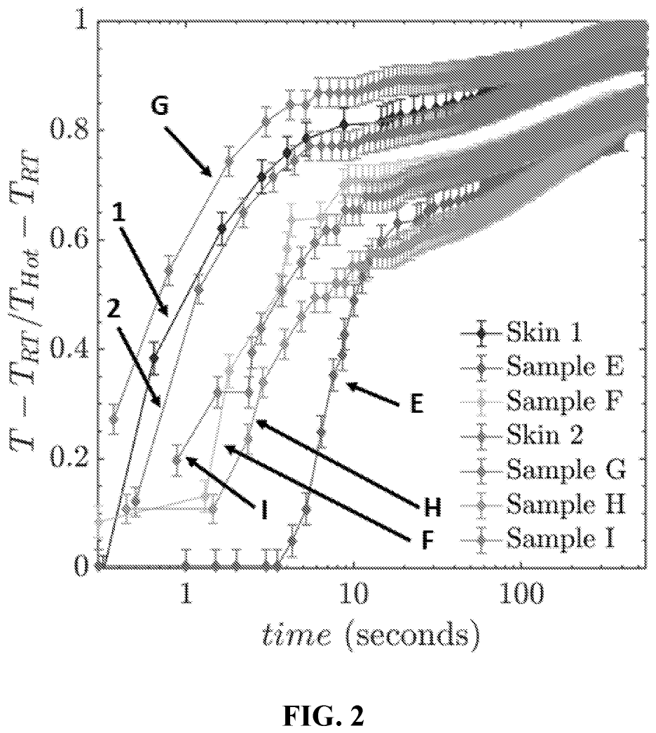

FIG. 1 is a graph showing normalized thermal response data for skin 0, and skin 0 with topical composition formulations A-D applied thereon. The temperatures are normalized using room temperature, T_{RT}=25° C., and T_{HOT}≈44° C. FIG. 2 is a graph showing normalized thermal response data for skin 1, skin 1 with topical composition formulations E or F applied thereon, skin 2, and skin 2 with topical composition formulations G, H or I applied thereon. The temperatures are normalized using room temperature, T_{RT}=25° C., and T_{HOT}≈44° C. For each data point in each figure, the error bars represent the minimum and maximum temperature accounting for the observed variance of T_{HOT}. In each figure, each data series (i.e., each skin and sample formulation) shows two distinct trends. At early times there is a steep increase in temperature before an inflection point, and after the inflection a more gradual increase occurs. All the samples display this behavior, but differ at the time the inflection is observed.

Although the present invention and its objects, features and advantages have been described in detail, other embodiments are encompassed by the invention. Finally, those skilled in the art should appreciate that they can readily use the disclosed conception and specific embodiments as a basis for designing or modifying other structures for carrying out the same purposes of the present invention without departing from the scope of the invention as defined by the appended claims.

1. A topical composition for protecting the skin of a mammal from the transfer of heat from a surface, the composition comprising a base formulation and additives, the additives comprising:
thermally-insulating nanoparticles and/or microparticles; and
one or more of a dimethicone, a cyclomethicone, and an amodimethicone.

2. The composition of claim 1, wherein the additives comprise two or more of the dimethicone, the cyclomethicone, and the amodimethicone.

3. The composition of claim 1, wherein the additives comprises the dimethicone, the cyclomethicone, and the amodimethicone.

4. The composition of claim 1, wherein the nanoparticles and/or microparticles comprise silica.

5. The composition of claim 1, wherein the topical composition comprises about 5 to about 35 v/v % of the thermally-insulating nanoparticles and/or microparticles.

6. The composition of claim 1, wherein the topical composition comprises about 2 to about 10 v/v % of the amodimethicone.

7. The composition of claim 1, wherein the topical composition comprises about 5 to about 10 v/v % of the cyclomethicone.

8. The composition of claim 1, wherein the topical composition comprises about 4 to about 10 v/v % of the dimethicone.

9. The composition of claim 1, wherein the nanoparticles and/or microparticles comprise a metal oxide.

10. The composition of claim 1, wherein the nanoparticles and/or microparticles comprise an insoluble ionic salt.

11. The composition of claim 1, wherein the nanoparticles and/or microparticles comprise a polymer or resin.

12. The composition of claim 1, wherein the topical composition comprises about 40 to about 80 v/v % of the base formulation.

13. A topical composition for protecting the skin of a mammal from the transfer of heat from a surface, the composition comprising:
a base formulation; and
one or more additives, the one or more additives comprising:
a silicone acrylate emulsion, the silicone acrylate emulsion comprising a blend of about 30% of acrylates/polytrimethylsiloxy-methacrylate copolymer anionic emulsion and laureth-1 phosphate.

14. The composition of claim 13, wherein the one or more additives further comprises thermally-insulating nanoparticles and/or microparticles.

15. The composition of claim 14, wherein the thermally-insulating nanoparticles and/or microparticles comprise silica.

16. The composition of claim 14, wherein the topical composition comprises about 5 to about 35 v/v % of the thermally-insulating nanoparticles and/or microparticles.

17. A topical composition for protecting the skin of a mammal from the transfer of heat from a surface, the composition comprising:
a base formulation; and
one or more additives, the one or more additives comprising:
a 60% non-ionic emulsion of a polydimethylsiloxane/vinyl copolymer having a viscosity of greater than 120×10⁶ mm²/s at 0.01 Hz, and having C12-13 Pareth-23 and C12-13 Pareth-3.

18. The composition of claim 17, wherein the one or more additives further comprises thermally-insulating nanoparticles and/or microparticles.

19. The composition of claim 18, wherein the thermally-insulating nanoparticles and/or microparticles comprise silica.

20. The composition of claim 18, wherein the topical composition comprises about 5 to about 35 v/v % of the thermally-insulating nanoparticles and/or microparticles.
